SEQUENCE LISTING (17 sequences)

SEQ ID NO: 1 - DNA, 794 bp, synthetic construct
  source 1..794: mol_type = other DNA
gaattcgccgccaccatgtacaggatgcagctgctgtcctgcatcgccctgagcctggccctggtgacaaattccgaggccaagcctagcggcagcgtggtggagcaggccgagggagtggagtgcgacttctcccccctgctgagcggcacccccccacaagtgtacaacttcaagagactggtgttcacaaactgtaattacaacctgaccaagctgctgtccctgttctccgtgaatgatttcacctgcagccagatctcccctgccgccatcgcctccaactgctactccagcctgatcctggattacttctcctaccccctgagcatgaagtccgatctgagcgtgagctccgccggccccatcagccagttcaactacaagcagtccttctccaaccctacctgtctgatcctggccaccgtgccccacaatctgaccaccatcaccaagcccctgaagtactcctacatcaataagtgtagcaggctgctgtccgacgatagaacagaggtgcctcagctggtgaatgccaaccagtacagcccctgcgtgagcatcgtgcctagcaccgtgtgggaggatggcgactactacaggaagcagctgtcccccctggagggcggcggatggctggttgcttccggcagcaccgtggccatgaccgagcagctgcagatgggcttcggcatcaccgtgcagtacggcacagacaccaatagcgtgtgtcctaagctggagttcgccaacgatacaaagatcgccagccagctgggcaattgagcggccgc

SEQ ID NO: 2 - protein, 256 aa, synthetic construct
MYRMQLLSCIALSLALVTNSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGN

SEQ ID NO: 3 - DNA, 722 bp, synthetic construct
  source 1..722: mol_type = other DNA
gaattcgccgccaccatgtacaggatgcagctgctgagctgtatcgccctgagcctggccctggtgaccaatagccaggccgagggcgtggagtgtgacttttcccctctgctgagcggcacccctcctcaggtgtacaatttcaagagactggtgttcacaaactgcaattacaacctgacaaagctgctgagcctgttctccgtgaatgacttcacatgcagccagatcagccccgccgccatcgccagcaactgctactcctccctgatcctggactacttctcctaccctctgtccatgaagagcgatctgagcgtgtccagcgccggccccatctcccagtttaactacaagcagtccttcagcaatcctacatgcctgatcctggccacagtgccccacaatctgaccaccatcaccaagcccctgaagtacagctacatcaacaagtgctccagactgctgagcgacgataggaccgaggtgcctcagctggtgaatgccaatcagtactccccttgtgtgtccatcgtgccttccacagtgtgggaggacggcgactactacaggaagcagctgtcccctctggagggcggcggctggctggttgctagcggatccaccgtggccatgaccgagcagctgcagatgggcttcggcatcacagtgcagtacggcacagacaccaacagcgtgtgtcccaagctgtgagcggccgc

SEQ ID NO: 4 - protein, 232 aa, synthetic construct
MYRMQLLSCIALSLALVTNSQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 5 - DNA, 1502 bp, synthetic construct
  source 1..1502: mol_type = other DNA
gaattcgccgccaccatgtacaggatgcagctgctgtcctgcatcgccctgagcctggccctggtgacaaattccgaggccaagcctagcggcagcgtggtggagcaggccgagggagtggagtgcgacttctcccccctgctgagcggcacccccccacaagtgtacaacttcaagagactggtgttcacaaactgtaattacaacctgaccaagctgctgtccctgttctccgtgaatgatttcacctgcagccagatctcccctgccgccatcgcctccaactgctactccagcctgatcctggattacttctcctaccccctgagcatgaagtccgatctgagcgtgagctccgccggccccatcagccagttcaactacaagcagtccttctccaaccctacctgtctgatcctggccaccgtgccccacaatctgaccaccatcaccaagcccctgaagtactcctacatcaataagtgtagcaggctgctgtccgacgatagaacagaggtgcctcagctggtgaatgccaaccagtacagcccctgcgtgagcatcgtgcctagcaccgtgtgggaggatggcgactactacaggaagcagctgtcccccctggagggcggcggatggctggttgcttccggcagcaccgtggccatgaccgagcagctgcagatgggcttcggcatcaccgtgcagtacggcacagacaccaatagcgtgtgtcctaagctggagttcgccaacgatacaaagatcgccagccagctgggcaatgaggccaagccctccggctccgtggtggagcaagccgagggcgtggagtgtgacttctcccctctgctgagcggaacccctcctcaggtgtacaactttaagagactggtcttcaccaactgcaactacaatctgacaaagctgctgagcctgttcagcgtgaacgacttcacctgtagccagatcagccccgccgccatcgctagcaactgttactcctccctgatcctggactacttttcctaccccctctccatgaagtccgacctgagcgtgtcctccgccggcccaatcagccagtttaactacaagcaaagctttagcaaccctacctgcctgatcctggctacagtgcctcacaatctgacaacaatcacaaagcctctgaagtacagctacatcaacaagtgcagcagactgctgtccgatgacaggaccgaggtgccccagctggtgaacgccaatcagtacagcccatgcgtgagcattgtgcctagcacagtgtgggaggacggcgattactacagaaagcagctgagccccctggagggaggcggctggctggttgcatccggctccaccgtggccatgacagagcagctgcaaatgggcttcggaatcaccgtgcaatacggcacagatacaaactccgtgtgccccaagctggagtttgccaacgataccaagatcgcctcccagctgggcaactgagcggccgc

SEQ ID NO: 6 - protein, 492 aa, synthetic construct
MYRMQLLSCIALSLALVTNSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGN

SEQ ID NO: 7 - DNA, 756 bp, synthetic construct
  source 1..756: mol_type = other DNA
gaattcgccgccaccatgcctctgctgctgctgctccccctgctgtgggccggagctctggctagggtgcagcccaccgagagcatcgtgaggttccccaatatcacaaatctgtgtcccttcggcgaggtgtttaacgccaccaggtttgcctccgtgtacgcctggaataggaagagaatcagcaattgtgtggccgactacagcgtgctgtacaattccgccagcttctccaccttcaagtgctacggcgtgagccccaccaagctgaatgacctgtgttttaccaatgtgtacgccgacagcttcgtgatcaggggcgatgaggtgaggcagatcgcccccggccagacaggcaagatcgccgattacaattacaagctgcctgatgattttaccggctgtgtgatcgcctggaatagcaataacctggatagcaaggtgggcggcaactacaattacctgtacagactgtttagaaagtccaacctgaagcccttcgagagggacatcagcaccgagatctaccaggccggctccacaccttgtaacggcgtggagggcttcaactgctactttcccctgcagagctacggcttccagcccaccaatggcgtgggctaccagccttacagagtggtggtgctgagctttgagctgctgcacgcccccgccaccgtgtgtggacctaagaagagcaccaatctggtgaagaataaggccgtgaactttaactttaatggcctgtgaaagctt

SEQ ID NO: 8 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKAVNFNFNGL

SEQ ID NO: 9 - DNA, 756 bp, synthetic construct
  source 1..756: mol_type = other DNA
gaattcgccgccaccatgcctctgctgctgctgctccccctgctgtgggccggagctctggctagggtgcagcctacagagtccatcgtgaggtttcctaacatcacaaacctgtgtccttttggcgaggtgtttaatgccacaagatttgccagcgtgtacgcctggaataggaagaggatcagcaattgcgtggccgactactccgtgctgtacaatagcgccagcttttccacctttaagtgctacggcgtgagccccacaaagctgaatgacctgtgttttaccaacgtgtacgccgacagctttgtgatcaggggcgacgaggtgagacagatcgcccccggccagaccggcaatatcgccgattacaactacaagctgcctgacgatttcacaggctgcgtgatcgcctggaatagcaacaatctggacagcaaggtgggcggcaactacaattacctgtacaggctgttcagaaagtccaacctgaagccctttgagagggacatctccacagagatctaccaggccggctccaccccctgtaatggcgtgaagggctttaactgttactttcccctgcagagctacggcttccagcccacctacggcgtgggctaccagccctacagagtggtggtgctgagcttcgagctgctgcacgcccctgccaccgtgtgcggacctaagaagagcaccaacctggtgaagaacaaggccgtgaactttaactttaatggcctgtgaaagctt

SEQ ID NO: 10 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKAVNFNFNGL

SEQ ID NO: 11 - DNA, 756 bp, synthetic construct
  source 1..756: mol_type = other DNA
gaattcgccgccaccatgcctctgctgctgctgctccccctgctgtgggccggagctctggctagggtgcagcccaccgagagcatcgtgaggttccccaatatcacaaatctgtgtcccttcggcgaggtgtttaacgccaccaggtttgcctccgtgtacgcctggaataggaagagaatcagcaattgtgtggccgactacagcgtgctgtacaattccgccagcttctccaccttcaagtgctacggcgtgagccccaccaagctgaatgacctgtgttttaccaatgtgtacgccgacagcttcgtgatcaggggcgatgaggtgaggcagatcgcccccggccagacaggcaagatcgccgattacaattacaagctgcctgatgattttaccggctgtgtgatcgcctggaatagcaataacctggatagcaaggtgggcggcaactacaattacagatacagactgtttagaaagtccaacctgaagcccttcgagagggacatcagcaccgagatctaccaggccggctccaagccttgtaacggcgtggagggcttcaactgctactttcccctgcagagctacggcttccagcccaccaatggcgtgggctaccagccttacagagtggtggtgctgagctttgagctgctgcacgcccccgccaccgtgtgtggacctaagaagagcaccaatctggtgaagaataaggccgtgaactttaactttaatggcctgtgaaagctt

SEQ ID NO: 12 - protein, 244 aa, synthetic construct
MPLLLLLPLLWAGALARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKAVNFNFNGL

SEQ ID NO: 13 - DNA, 731 bp, synthetic construct
  source 1..731: mol_type = other DNA
gaattcgccgccaccatgcccctgctgctgctgctccctctgctgtgggccggcgctctggctagagtgcagcctacagagagcatcgtgaggttccctaatatcacaaacctgtgcccttttgacgaggtgttcaacgccacaaggtttgcctccgtgtacgcctggaacagaaagagaatcagcaattgtgtggccgattacagcgtgctgtacaatctggccccctttttcacatttaagtgttacggcgtgtcccccaccaagctgaatgatctgtgcttcaccaacgtgtacgccgacagctttgtgatcagaggcgacgaggtgagacagatcgcccctggccagaccggcaacatcgccgattacaactacaagctgcccgatgactttaccggctgcgtgatcgcctggaactccaacaagctggacagcaaggtgtccggcaactacaactacctgtacaggctgttcaggaagtccaatctgaagcctttcgagagagatatctccacagagatctaccaggccggcaacaagccctgcaatggcgtggccggctttaattgttactttcctctgcgaagctactcctttagacctacctacggcgtgggccaccagccttacagagtggtggtgctgtcctttgagctgctgcacgcccctgccacagtgtgtggccccaagaagtccaccaacctggtgaagaacaagtgagcggccgc

SEQ ID NO: 14 - protein, 235 aa, synthetic construct
MPLLLLLPLLWAGALARVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 15 - protein, 8 aa, synthetic construct
DYKDDDDK

SEQ ID NO: 16 - protein, 12 aa, synthetic construct
PDRVRAVSHWSS

SEQ ID NO: 17 - skipped

(19) United States
(12) Patent Application Publication
(10) Pub. No.: US 2026/0158134 A1
(43) Pub. Date: Jun. 11, 2026

(54) COMPOSITIONS SUITABLE FOR USE IN A METHOD FOR ELICITING CROSS-PROTECTIVE IMMUNITY AGAINST CORONAVIRUSES

(71) Applicant: King Abdullah University of Science and Technology, Thuwal (SA)

(72) Inventors: Jasdave Chahal, Woburn, MA (US); Justine McPartlan, Woburn, MA (US); Magnus Rueping, Thuwal (SA); Jorg Eppinger, Neufahrn bei Freising (DE); Dominik Renn, Thuwal (SA); Poulami Talukder, Woburn, MA (US)

(21) Appl. No.: 18/707,694

(22) PCT Filed: Nov. 7, 2023
(86) PCT No.: PCT/IB2022/060699

§ 371 Date: May 6, 2024

Related U.S. Application Data
(60) Provisional application No. 63/276,302, filed on Nov. 5, 2021.

(51) Int. Cl. A61K 39/215; A61K 39/00; A61P 37/04

(52) U.S. Cl. CPC A61K 39/215; A61P 37/04; A61K 2039/53; A61K 2039/54; A61K 2039/543; A61K 2039/645; A61K 2039/70

(57) ABSTRACT

Immunogenic compositions and methods of use thereof, for eliciting an immune response against multiple coronaviruses, using a single vaccine composition are described. The compositions include an antigen from more than one pathogen, for example, more than one member of the β-coronavirus family, for example, SARS-CoV and MERS-CoV. Exemplary antigens include the receptor binding domain (RBD) of the coronavirus spike protein or a fragment thereof. The disclosed compositions are administered to a subject in need therefore, to generate an immune response against more than one pathogen, represented by the source of the antigens in the construct.

### CROSS-REFERENCE TO RELATED APPLICATIONS

This application claims the benefit of and priority to U.S. Ser. No. 63/276,302, filed on Nov. 5, 2021, which is incorporated by reference herein in its entirety.

### REFERENCE TO SEQUENCE LISTING

The Sequence Listing submitted as an xml file named “KAUST_2022_029_02_PCT.xml,” created on Nov. 3, 2022, and having a size of 23,083 bytes is hereby incorporated by reference pursuant to 37 C.F.R. § 1.834 (c) (1).

### FIELD OF THE INVENTION

This invention is generally in the field of immunogenic compositions and methods of use thereof to elicit cross-protective immunity against coronaviruses.

### BACKGROUND OF THE INVENTION

Coronaviruses are a significant threat to human health, and multiple serologically distinct species exist that are capable of infecting and causing disease in humans and animals (Poon, et al. Nature Medicine 2020, 26 (3), 317-319; Khan, et al. Turk J Emerg Med 2020, 20 (2), 55-62). Most recently, the COVID-19 pandemic has promoted an interest in development of a pancoronavirus vaccine, as vaccines are the most effective means of controlling outbreaks and protecting populations against disease (Excler, et al. Nature Medicine 2021, 27 (4), 591-600). While COVID-19 vaccine candidates (Krammer, Nature 2020, 586 (7830), 516-527; Chakraborty, et al. Advanced Drug Delivery Reviews 2021, 172, 314-338), have achieved licensure, most focus on the SARS-CoV-2 full length Spike protein, protection is limited to SARS-CoV-2, and long-term durability of that protection remains unknown as documented cases of re-infection accumulate, and vaccine-resistant variants arise. The emergence of the Delta strain and “breakthrough cases” affecting vaccinated individuals demonstrate the need for a broadly protective coronavirus vaccine platform (Shastri, et al. Frontiers in Medicine 2021, 8 (1379)). SARS-CoV-2 is only one of seven coronavirus strains, including SARS-CoV-1, and the Middle East Respiratory Syndrome Coronavirus (MERS-CoV) threatening human health (Zhu, et al. Respiratory Research 2020, 21 (1), 224).

The Kingdom of Saudi Arabia was the site of the first MERS-CoV infection and has experienced the worst outbreaks of the virus. According to the World Health Organization (WHO), by October 2020 MERS-CoV-related fatalities have reached 866 globally, with 788 in Saudi Arabia alone. So far, MERS-CoV infections have reached a total of 2519 worldwide, including 2077 infections in Saudi Arabia (World Health Organization Middle East respiratory syndrome coronavirus (MERS-CoV)—Situation update. emro.who.int/health-topics/mers-cov/mers-outbreaks (accessed 30 Oct. 2020)). The high fatality rate (approx. 34.4%) and its persistent spread in animal pools, particularly dromedary camels, have generated significant public health concerns (World Health Organization Middle East respiratory syndrome coronavirus (MERS-CoV)—The Kingdom of Saudi Arabia. emro.who.int/health-topics/mers-cov/mers-outbreaks (accessed 30 Oct. 2020)). However, so far, no licensed prophylactic or therapeutic agent against MERS-CoV is available (Nature Research KAIMRC a strong player in quest for a MERS-CoV vaccine. www.nature.com/articles/d42473-020-00067-2: Xu, et al. Emerg Microbes Infect 2019, 8 (1), 841-856).

Beyond these recent outbreaks, other circulating coronaviruses are known to cause respiratory tract infections, accounting for up to 30% of all common colds (Liu, D. X.; Liang, J. Q.: Fung, T. S., Human Coronavirus-229E, -OC43, -NL63, and -HKU1 (Coronaviridae). Encyclopedia of Virology 2021, 428-440). With over 6,000 coronaviruses strains lying dormant in bats alone, the COVID-19 pandemic is neither the first, nor will it be the last, to threaten human life. Today there is no vaccine available that provides protection across different families of coronaviruses. A broadly protective vaccine comprising RBDs from multiple coronaviruses has the potential to protect the population against the current COVID-19 pandemic as well as possible future coronavirus outbreaks.

It is an object of the present invention to provide broadly protective vaccine compositions for eliciting neutralizing antibodies effective against different strains of coronaviruses in a subject in need thereof.

It is also an object of the present invention to provide a method for making and using the broadly protective vaccine compositions against a coronavirus infection in a subject in need thereof.

### SUMMARY OF THE INVENTION

Immunogenic compositions and methods of use thereof, are provided. The disclosed methods and compositions serve to protect a subject (for example, a person) against multiple coronaviruses, using a single vaccine composition. The compositions are immunogenic compositions which include an antigen from more than one pathogen, for example, more than one member of the β-coronavirus family for example, SARS-CoV and MERS-CoV. The compositions preferably include a nucleic acid molecule construct such as an RNA construct, which encodes antigens from more than one family member of the β-coronavirus family. A preferred antigen is the receptor binding domain (RBD) of the coronavirus spike protein, or a fragment of the spike protein including the RDB. Preferably, full length spike protein is not used. In one embodiment the construct encodes RBD only, of more than one coronavirus. However, the construct can also encode immunogenic portions of the N or E protein. In some embodiments, the compositions include an adjuvant.

The disclosed compositions are administered to a subject in need thereof, to generate an immune response against more than one pathogen, represented by the source of the antigens in the construct. Preferably, the immune response generated is a neutralizing immune response.

The disclosed methods minimize the number of RNA constructs included in a vaccine formulation, allow for lower dosage, particularly in a replicon vaccine, as the nonstructural components of the replicon typically contribute a greater portion of the total mass of the RNA than the encoded antigens.

### BRIEF DESCRIPTION OF THE DRAWINGS

FIG. 1 is a schematic design of an exemplary synthetic polypeptide, N-terminus to the left and C-terminus to the right, including a signal peptide and two RBD segments from different betacoronaviruses. A C-terminal FLAG tag is included.

FIGS. 2A-2B are immunoblots of lysates from cells transfected with RNA encoding the polypeptide depicted in FIG. 1 (B.1.617.2-MERS RBD dimer). Lanes 1 and 2 represent two different lots of the replicon RNA, and lane C, untransfected cells (negative control). The same lysates were immobilized on a PVDF membrane and probed with antibody specific for SARS-CoV-2 (FIG. 2A) or MERS-CoV (FIG. 2B). GAPDH is used as loading control

FIGS. 3A-3B are graphs showing endpoint titers of anti-B.1.351 RBD IgG (FIG. 3A) and anti-MER RBD IgG (FIG. 3B) in sera of mice immunized with an RNA vaccine encoding B.1.351 Full Length Spike, MERS-CoV RBD, or B.1.617.2-MERS RBD dimer (depicted in FIG. 1), and a negative control.

FIGS. 4A-4C are graphs showing endpoint titers of anti-SARS-CoV-2 Beta RBD IgG (FIG. 4A), anti-SARS-CoV-2 Delta RBD IgG (FIG. 4B), and anti-MERS-CoV RBD (FIG. 4C) in sera of mice immunized with a mixture of the B.1.617.2-MERS RBD dimer (FIG. 1) RNA and an additional vaccine RNA encoding the SARS-CoV-2 Spike protein co-formulated for administration as a single nanoparticle product (“Trivalent NP”).

FIGS. 5A-5C are graphs showing endpoint titers of anti-SARS-CoV-2 Beta RBD IgG (FIG. 5A), anti-SARS-CoV-2 Delta RBD IgG (FIG. 5B), and anti-MERS-CoV RBD IgG (FIG. 5C) in sera of mice immunized with Trivalent RNA vaccines at Day 20, 34, and 48 post vaccination in a prime/boost regimen (n=6). The Trivalent RNA vaccine included one RNA molecule encoding the B.1.617.2-MERS RBD dimer, and another encoding the full-length SARS-CoV-2 variant B.1.351 (Beta) full-length Spike protein.

### DETAILED DESCRIPTION OF THE INVENTION

The present invention is an improvement to current vaccine designs, as it allows for vaccination against multiple coronaviruses with a single RNA molecule or recombinant protein. The disclosed compositions are based on a rationally engineered vaccine protein antigen that comprises immunogenic protein domains from more than one strain of coronavirus. This is hereby referred to as the ‘multimer’ principle. This principle is applied by the production of a fusion protein composed of two or more RBDs from different strains of coronavirus either with or without a linker. The proposed product could be in the form of mRNA, replicon RNA, DNA, or a recombinant protein.

### I. Definitions

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount which is able to treat one or more symptoms of a disease or disorder, reverse the progression of one or more symptoms of a disease or disorder, halt the progression of one or more symptoms of a disease or disorder, or prevent the occurrence of one or more symptoms of a disease or disorder in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salt” is art-recognized, and includes relatively non-toxic, inorganic, and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine: mono-, di- or trihydroxyalkylamines such as mono-, di-, and triethanolamine; amino acids, such as arginine and lysine: guanidine: N-methylglucosamine: N-methylglucamine: L-glutamine: N-methylpiperazine: morpholine: ethylenediamine: N-benzylphenethylamine: etc.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including therapeutic agents may inhibit or reduce one or more markers of a disease or disorder in a subject by about 10%, 20%, 30%, 40%, 50%, 75%, 85%, 90%, 95%, or 99% from the activity and/or quantity of the same marker in subjects that did not receive, or were not treated with the compositions. In some embodiments, the inhibition and reduction are compared according to the level of mRNAs, proteins, cells, tissues, and organs.

The terms “treating” or “retarding development of” in the context of a disease or disorder mean to ameliorate, reduce or otherwise stop a disease, disorder or condition from occurring or progressing in an animal which may be predisposed to the disease, disorder and/or condition but has not yet been diagnosed as having it: inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with a coronavirus infection are mitigated or eliminated, including, but are not limited to, reducing and/or inhibiting the syncytial formation and lung damage, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The term “adjuvant” refers to a compound or mixture that enhances an immune response.

The term “immunogenic composition” or “composition” means that the composition can induce an immune response and is therefore antigenic. By “immune response” means any reaction by the immune system. These reactions include the alteration in the activity of an organism's immune system in response to an antigen and can involve, for example, antibody production, induction of cell-mediated immunity, complement activation, or development of immunological tolerance.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

### II. Compositions

Compositions for eliciting an immune against multiple coronaviruses with a single RNA molecule or recombinant protein (herein, heterogenous multimer antigen) include (i) an RNA molecule encoding antigens from multiple coronaviruses or (ii) a fusion peptide containing antigens from multiple coronaviruses (heterogeneous antigens). The heterogenous multimer antigen compositions are immunogenic against multiple coronaviruses based on the source of each antigen, and can include a carrier such as anionic liposome, dendrimer, polynucleotide, synthetic nanoparticle, modified dendrimer nanoparticle, microgel, hydrogel, etc. The disclosed compositions are advantageous over compositions providing the antigens on separate mRNA's because a formulation with 2 different RNAs has more nucleic acid material than 1 RNA that encodes 1 ORF (open reading frame). 1 ORF is more efficient and requires less sequence space than 2 ORFs. Immunological benefit of the disclosed compositions it that it presents 2 antigens linked so that the structure presents 2 antigenic sites in multimeric format is more likely to stimulate AB response because antigens presented in patterned formats better stimulate B-cell receptor responses.

### A. Multimer Antigens

Antigens can be or can include, for example, peptides, proteins, polysaccharides, saccharides, lipids, nucleic acids, small molecules (alone or with a hapten), or combinations thereof. The multimer antigen preferably is a polypeptide or a nucleic acid encoding a polypeptide (mRNA) including: (i) a signal peptide at the N-terminus (for example, tissue plasminogen activator (tPA) signal sequence, or an extended tPA, or a signal peptide derived from a full-length coronavirus spike protein); (ii) the receptor-binding domain (RBD) of a first coronavirus Spike protein downstream of the signal peptide: (iii) an optional linker: (iv) the receptor-binding domain of a second coronavirus different from the first fused to the preceding receptor-binding domain; and optionally, (iv) an affinity tag such FLAG-tag. An exemplary construct of a polypeptide or a nucleic acid (mRNA) encoding a polypeptide of a multimer antigen is shown in FIG. 1.

The disclosed constructs preferably do not include a dimer of RBD from the same coronavirus or encode a fusion protein with two identical RBD's from the same coronavirus (homogeneous RBDs), back-to-back, on the same construct. Preferably, the disclosed constructs include RDBs from different coronaviruses, which eliminates the need for preparing separate immunogenic compositions for each coronavirus.

Exemplary nucleic acids and amino acid sequences are shown below.

Nucleotide Sequence of MERS-COV Spike RBD Antigen; 256 AAs,
MW = 27.98 kDa (optimized for CHO)
(SEQ ID NO: 1)
gaattcgccgccaccATGTACAGGATGCAGCTGCTGTCCTGCATCGCCCTG
AGCCTGGCCCTGGTGACAAATTCCGAGGCCAAGCCTAGCGGCAGC
GTGGTGGAGCAGGCCGAGGGAGTGGAGTGCGACTTCTCCCCCCTG
CTGAGCGGCACCCCCCCACAAGTGTACAACTTCAAGAGACTGGTG
TTCACAAACTGTAATTACAACCTGACCAAGCTGCTGTCCCTGTTCT
CCGTGAATGATTTCACCTGCAGCCAGATCTCCCCTGCCGCCATCG
CCTCCAACTGCTACTCCAGCCTGATCCTGGATTACTTCTCCTACCC
CCTGAGCATGAAGTCCGATCTGAGCGTGAGCTCCGCCGGCCCCAT
CAGCCAGTTCAACTACAAGCAGTCCTTCTCCAACCCTACCTGTCTG
ATCCTGGCCACCGTGCCCCACAATCTGACCACCATCACCAAGCCC
CTGAAGTACTCCTACATCAATAAGTGTAGCAGGCTGCTGTCCGAC
GATAGAACAGAGGTGCCTCAGCTGGTGAATGCCAACCAGTACAG
CCCCTGCGTGAGCATCGTGCCTAGCACCGTGTGGGAGGATGGCGA
CTACTACAGGAAGCAGCTGTCCCCCCTGGAGGGCGGCGGATGGCT
GGTTGCTTCCGGCAGCACCGTGGCCATGACCGAGCAGCTGCAGAT
GGGCTTCGGCATCACCGTGCAGTACGGCACAGACACCAATAGCGT
GTGTCCTAAGCTGGAGTTCGCCAACGATACAAAGATCGCCAGCCA
GCTGGGCAATtgagcggccgc
Amino Acid Sequence of MERS-COV Spike RBD Antigen; 256 AAs,
MW = 27.98 kDa; Bold letters: secretion-tag
(SEQ ID NO: 2)
MYRMQLLSCIALSLALVTNSEAKPSGSVVEQAEGVECDFSPLLSGT
PPQVYNFKRLVFTNCNYNLTKLLSLFSVNDFTCSQISPAAIASNCYSS
LILDYFSYPLSMKSDLSVSSAGPISQFNYKQSFSNPTCLILATVPHNLT
TITKPLKYSYINKCSRLLSDDRTEVPQLVNANQYSPCVSIVPSTVWED
GDYYRKQLSPLEGGGWLVASGSTVAMTEQLQMGFGITVQYGTDTN
SVCPKLEFANDTKIASQLGN.
Nucleotide Sequence of MERS-COV Spike RBD 2 Antigen
(optimized for CHO)
(SEQ ID NO: 3)
gaattcgccgccaccATGTACAGGATGCAGCTGCTGAGCTGTATCG
CCCTGAGCCTGGCCCTGGTGACCAATAGCCAGGCCGAGGGCGTGG
AGTGTGACTTTTCCCCTCTGCTGAGCGGCACCCCTCCTCAGGTGTA
CAATTTCAAGAGACTGGTGTTCACAAACTGCAATTACAACCTGAC
AAAGCTGCTGAGCCTGTTCTCCGTGAATGACTTCACATGCAGCCA
GATCAGCCCCGCCGCCATCGCCAGCAACTGCTACTCCTCCCTGAT
CCTGGACTACTTCTCCTACCCTCTGTCCATGAAGAGCGATCTGAGC
GTGTCCAGCGCCGGCCCCATCTCCCAGTTTAACTACAAGCAGTCC
TTCAGCAATCCTACATGCCTGATCCTGGCCACAGTGCCCCACAAT
CTGACCACCATCACCAAGCCCCTGAAGTACAGCTACATCAACAAG
TGCTCCAGACTGCTGAGCGACGATAGGACCGAGGTGCCTCAGCTG
GTGAATGCCAATCAGTACTCCCCTTGTGTGTCCATCGTGCCTTCCA
CAGTGTGGGAGGACGGCGACTACTACAGGAAGCAGCTGTCCCCTC
TGGAGGGCGGCGGCTGGCTGGTTGCTAGCGGATCCACCGTGGCCA
TGACCGAGCAGCTGCAGATGGGCTTCGGCATCACAGTGCAGTACG
GCACAGACACCAACAGCGTGTGTCCCAAGCTGtgagcggccgc.
Amino Acid of MERS-COV Spike RBD 2 Antigen; 232 AAs,
MW = 25.51 kDa; Bold letters: secretion-tag
(SEQ ID NO: 4)
MYRMQLLSCIALSLALVTNSQAEGVECDFSPLLSGTPPQVYNFKRL
VFTNCNYNLTKLLSLFSVNDFTCSQISPAAIASNCYSSLILDYFSYPLS
MKSDLSVSSAGPISQFNYKQSFSNPTCLILATVPHNLTTITKPLKYSYI
NKCSRLLSDDRTEVPQLVNANQYSPCVSIVPSTVWEDGDYYRKQLSP
LEGGGWLVASGSTVAMTEQLQMGFGITVQYGTDTNSVCPKL.
Nucleotide Sequence of MERS-COV Spike RBD Dimer Antigen
(optimized for CHO):
(SEQ ID NO: 5)
gaattcgccgccaccATGTACAGGATGCAGCTGCTGTCCTGCATCGCCCTG
AGCCTGGCCCTGGTGACAAATTCCGAGGCCAAGCCTAGCGGCAGC
GTGGTGGAGCAGGCCGAGGGAGTGGAGTGCGACTTCTCCCCCCTG
CTGAGCGGCACCCCCCCACAAGTGTACAACTTCAAGAGACTGGTG
TTCACAAACTGTAATTACAACCTGACCAAGCTGCTGTCCCTGTTCT
CCGTGAATGATTTCACCTGCAGCCAGATCTCCCCTGCCGCCATCG
CCTCCAACTGCTACTCCAGCCTGATCCTGGATTACTTCTCCTACCC
CCTGAGCATGAAGTCCGATCTGAGCGTGAGCTCCGCCGGCCCCAT
CAGCCAGTTCAACTACAAGCAGTCCTTCTCCAACCCTACCTGTCTG
ATCCTGGCCACCGTGCCCCACAATCTGACCACCATCACCAAGCCC
CTGAAGTACTCCTACATCAATAAGTGTAGCAGGCTGCTGTCCGAC
GATAGAACAGAGGTGCCTCAGCTGGTGAATGCCAACCAGTACAG
CCCCTGCGTGAGCATCGTGCCTAGCACCGTGTGGGAGGATGGCGA
CTACTACAGGAAGCAGCTGTCCCCCCTGGAGGGCGGCGGATGGCT
GGTTGCTTCCGGCAGCACCGTGGCCATGACCGAGCAGCTGCAGAT
GGGCTTCGGCATCACCGTGCAGTACGGCACAGACACCAATAGCGT
GTGTCCTAAGCTGGAGTTCGCCAACGATACAAAGATCGCCAGCCA
GCTGGGCAATGAGGCCAAGCCCTCCGGCTCCGTGGTGGAGCAAG
CCGAGGGCGTGGAGTGTGACTTCTCCCCTCTGCTGAGCGGAACCC
CTCCTCAGGTGTACAACTTTAAGAGACTGGTCTTCACCAACTGCA
ACTACAATCTGACAAAGCTGCTGAGCCTGTTCAGCGTGAACGACT
TCACCTGTAGCCAGATCAGCCCCGCCGCCATCGCTAGCAACTGTT
ACTCCTCCCTGATCCTGGACTACTTTTCCTACCCCCTCTCCATGAA
GTCCGACCTGAGCGTGTCCTCCGCCGGCCCAATCAGCCAGTTTAA
CTACAAGCAAAGCTTTAGCAACCCTACCTGCCTGATCCTGGCTAC
AGTGCCTCACAATCTGACAACAATCACAAAGCCTCTGAAGTACAG
CTACATCAACAAGTGCAGCAGACTGCTGTCCGATGACAGGACCGA
GGTGCCCCAGCTGGTGAACGCCAATCAGTACAGCCCATGCGTGAG
CATTGTGCCTAGCACAGTGTGGGAGGACGGCGATTACTACAGAAA
GCAGCTGAGCCCCCTGGAGGGAGGCGGCTGGCTGGTTGCATCCGG
CTCCACCGTGGCCATGACAGAGCAGCTGCAAATGGGCTTCGGAAT
CACCGTGCAATACGGCACAGATACAAACTCCGTGTGCCCCAAGCT
GGAGTTTGCCAACGATACCAAGATCGCCTCCCAGCTGGGCAACtga
gcggccgc.
Amino Acid Sequence of MERS-COV Spike RBD Antigen; 492 AAs,
MW-53.74 kDa; Bold letters: secretion-tag
(SEQ ID NO: 6)
MYRMQLLSCIALSLALVTNSEAKPSGSVVEQAEGVECDFSPLLSGT
PPQVYNFKRLVFTNCNYNLTKLLSLFSVNDFTCSQISPAAIASNCYSS
LILDYFSYPLSMKSDLSVSSAGPISQFNYKQSFSNPTCLILATVPHNLT
TITKPLKYSYINKCSRLLSDDRTEVPQLVNANQYSPCVSIVPSTVWED
GDYYRKQLSPLEGGGWLVASGSTVAMTEQLQMGFGITVQYGTDTN
SVCPKLEFANDTKIASQLGNEAKPSGSVVEQAEGVECDFSPLLSGTPP
QVYNFKRLVFTNCNYNLTKLLSLFSVNDFTCSQISPAAIASNCYSSLIL
DYFSYPLSMKSDLSVSSAGPISQFNYKQSFSNPTCLILATVPHNLTTIT
KPLKYSYINKCSRLLSDDRTEVPQLVNANQYSPCVSIVPSTVWEDGD
YYRKQLSPLEGGGWLVASGSTVAMTEQLQMGFGITVQYGTDTNSV
CPKLEFANDTKIASQLGN.
Nucleotide Sequence of SARS-COV-2 Wuhan Spike RBD Antigen;
244 AAs, MW = 27.29 kDa (optimized for CHO):
(SEQ ID NO: 7)
gaattcgccgccaccATGCCTCTGCTGCTGCTGCTCCCCCTGCTGTGGGCC
GGAGCTCTGGCTAGGGTGCAGCCCACCGAGAGCATCGTGAGGTTC
CCCAATATCACAAATCTGTGTCCCTTCGGCGAGGTGTTTAACGCC
ACCAGGTTTGCCTCCGTGTACGCCTGGAATAGGAAGAGAATCAGC
AATTGTGTGGCCGACTACAGCGTGCTGTACAATTCCGCCAGCTTC
TCCACCTTCAAGTGCTACGGCGTGAGCCCCACCAAGCTGAATGAC
CTGTGTTTTACCAATGTGTACGCCGACAGCTTCGTGATCAGGGGC
GATGAGGTGAGGCAGATCGCCCCCGGCCAGACAGGCAAGATCGC
CGATTACAATTACAAGCTGCCTGATGATTTTACCGGCTGTGTGATC
GCCTGGAATAGCAATAACCTGGATAGCAAGGTGGGCGGCAACTA
CAATTACCTGTACAGACTGTTTAGAAAGTCCAACCTGAAGCCCTT
CGAGAGGGACATCAGCACCGAGATCTACCAGGCCGGCTCCACAC
CTTGTAACGGCGTGGAGGGCTTCAACTGCTACTTTCCCCTGCAGA
GCTACGGCTTCCAGCCCACCAATGGCGTGGGCTACCAGCCTTACA
GAGTGGTGGTGCTGAGCTTTGAGCTGCTGCACGCCCCCGCCACCG
TGTGTGGACCTAAGAAGAGCACCAATCTGGTGAAGAATAAGGCC
GTGAACTTTAACTTTAATGGCCTGtgaaagctt.
Amino Acid Sequence of SARS-COV-2 Wuhan Spike RBD Antigen;
244 AAs, MW = 27.29 kDa; Bold letters = secretion-tag
(SEQ ID NO: 8)
MPLLLLLPLLWAGALARVQPTESIVRFPNITNLCPFGEVFNATRFAS
VYAWNRKRISNCVADYSVLYNSASFSTFKCYGVSPTKLNDLCFTNV
YADSFVIRGDEVRQIAPGQTGKIADYNYKLPDDFTGCVIAWNSNNLD
SKVGGNYNYLYRLFRKSNLKPFERDISTEIYQAGSTPCNGVEGFNCY
FPLQSYGFQPTNGVGYQPYRVVVLSFELLHAPATVCGPKKSTNLVKN
KAVNFNFNGL.
Nucleotide Sequence of SARS-COV-2 Beta (B.1.351) Variant Spike
RBD Antigen (optimized for CHO):
(SEQ ID NO: 9)
gaattcgccgccaccATGCCTCTGCTGCTGCTGCTCCCCCTGCTGTGGGCC
GGAGCTCTGGCTAGGGTGCAGCCTACAGAGTCCATCGTGAGGTTT
CCTAACATCACAAACCTGTGTCCTTTTGGCGAGGTGTTTAATGCCA
CAAGATTTGCCAGCGTGTACGCCTGGAATAGGAAGAGGATCAGC
AATTGCGTGGCCGACTACTCCGTGCTGTACAATAGCGCCAGCTTT
TCCACCTTTAAGTGCTACGGCGTGAGCCCCACAAAGCTGAATGAC
CTGTGTTTTACCAACGTGTACGCCGACAGCTTTGTGATCAGGGGC
GACGAGGTGAGACAGATCGCCCCCGGCCAGACCGGCAATATCGC
CGATTACAACTACAAGCTGCCTGACGATTTCACAGGCTGCGTGAT
CGCCTGGAATAGCAACAATCTGGACAGCAAGGTGGGCGGCAACT
ACAATTACCTGTACAGGCTGTTCAGAAAGTCCAACCTGAAGCCCT
TTGAGAGGGACATCTCCACAGAGATCTACCAGGCCGGCTCCACCC
CCTGTAATGGCGTGAAGGGCTTTAACTGTTACTTTCCCCTGCAGA
GCTACGGCTTCCAGCCCACCTACGGCGTGGGCTACCAGCCCTACA
GAGTGGTGGTGCTGAGCTTCGAGCTGCTGCACGCCCCTGCCACCG
TGTGCGGACCTAAGAAGAGCACCAACCTGGTGAAGAACAAGGCC
GTGAACTTTAACTTTAATGGCCTGtgaaagctt.
Amino Acid Sequence of SARS-COV-2 Beta (B.1.351) Variant Spike
RBD Antigen; 244 AAs, MW = 27.33 kDa, Bold letters: secretion-tag:
(SEQ ID NO: 10)
MPLLLLLPLLWAGALARVQPTESIVRFPNITNLCPFGEVFNATRFAS
VYAWNRKRISNCVADYSVLYNSASFSTFKCYGVSPTKLNDLCFTNV
YADSFVIRGDEVRQIAPGQTGNIADYNYKLPDDFTGCVIAWNSNNLD
SKVGGNYNYLYRLFRKSNLKPFERDISTEIYQAGSTPCNGVKGFNCY
FPLQSYGFQPTYGVGYQPYRVVVLSFELLHAPATVCGPKKSTNLVKN
KAVNFNFNGL.
Nucleotide Sequence of humanized SARS-COV-2 Delta (B.1.617.2)
Variant Spike RBD Antigen (optimized for CHO)
(SEQ ID NO: 11)
gaattcgccgccaccATGCCTCTGCTGCTGCTGCTCCCCCTGCTGTGGGCC
GGAGCTCTGGCTAGGGTGCAGCCCACCGAGAGCATCGTGAGGTTC
CCCAATATCACAAATCTGTGTCCCTTCGGCGAGGTGTTTAACGCC
ACCAGGTTTGCCTCCGTGTACGCCTGGAATAGGAAGAGAATCAGC
AATTGTGTGGCCGACTACAGCGTGCTGTACAATTCCGCCAGCTTC
TCCACCTTCAAGTGCTACGGCGTGAGCCCCACCAAGCTGAATGAC
CTGTGTTTTACCAATGTGTACGCCGACAGCTTCGTGATCAGGGGC
GATGAGGTGAGGCAGATCGCCCCCGGCCAGACAGGCAAGATCGC
CGATTACAATTACAAGCTGCCTGATGATTTTACCGGCTGTGTGATC
GCCTGGAATAGCAATAACCTGGATAGCAAGGTGGGCGGCAACTA
CAATTACAGATACAGACTGTTTAGAAAGTCCAACCTGAAGCCCTT
CGAGAGGGACATCAGCACCGAGATCTACCAGGCCGGCTCCAAGC
CTTGTAACGGCGTGGAGGGCTTCAACTGCTACTTTCCCCTGCAGA
GCTACGGCTTCCAGCCCACCAATGGCGTGGGCTACCAGCCTTACA
GAGTGGTGGTGCTGAGCTTTGAGCTGCTGCACGCCCCCGCCACCG
TGTGTGGACCTAAGAAGAGCACCAATCTGGTGAAGAATAAGGCC
GTGAACTTTAACTTTAATGGCCTGtgaaagctt.
Amino Acid Sequence of SARS-COV-2 Delta (B.1.617.2) Variant
Spike RBD Antigen; 244 AAs, MW = 27.36 kDa; Bold letters: secretion-tag
(SEQ ID NO: 12)
MPLLLLLPLLWAGALARVQPTESIVRFPNITNLCPFGEVFNATRFAS
VYAWNRKRISNCVADYSVLYNSASFSTFKCYGVSPTKLNDLCFTNV
YADSFVIRGDEVRQIAPGQTGKIADYNYKLPDDFTGCVIAWNSNNLD
SKVGGNYNYRYRLFRKSNLKPFERDISTEIYQAGSKPCNGVEGFNCY
FPLQSYGFQPTNGVGYQPYRVVVLSFELLHAPATVCGPKKSTNLVKN
KAVNFNFNGL.
Nucleotide Sequence of SARS-COV-2 Omicron Variant (B.1.1.529,
BA.1) Spike RBD Antigen:
(SEQ ID NO: 13)
GAATTCGCCGCCACCATGCCCCTGCTGCTGCTGCTCCCTCTGCTGT
GGGCCGGCGCTCTGGCTAGAGTGCAGCCTACAGAGAGCATCGTG
AGGTTCCCTAATATCACAAACCTGTGCCCTTTTGACGAGGTGTTCA
ACGCCACAAGGTTTGCCTCCGTGTACGCCTGGAACAGAAAGAGA
ATCAGCAATTGTGTGGCCGATTACAGCGTGCTGTACAATCTGGCC
CCCTTTTTCACATTTAAGTGTTACGGCGTGTCCCCCACCAAGCTGA
ATGATCTGTGCTTCACCAACGTGTACGCCGACAGCTTTGTGATCA
GAGGCGACGAGGTGAGACAGATCGCCCCTGGCCAGACCGGCAAC
ATCGCCGATTACAACTACAAGCTGCCCGATGACTTTACCGGCTGC
GTGATCGCCTGGAACTCCAACAAGCTGGACAGCAAGGTGTCCGGC
AACTACAACTACCTGTACAGGCTGTTCAGGAAGTCCAATCTGAAG
CCTTTCGAGAGAGATATCTCCACAGAGATCTACCAGGCCGGCAAC
AAGCCCTGCAATGGCGTGGCCGGCTTTAATTGTTACTTTCCTCTGC
GAAGCTACTCCTTTAGACCTACCTACGGCGTGGGCCACCAGCCTT
ACAGAGTGGTGGTGCTGTCCTTTGAGCTGCTGCACGCCCCTGCCA
CAGTGTGTGGCCCCAAGAAGTCCACC
AACCTGGTGAAGAACAAGTGAGCGGCCGC.
SARS-COV-2 Omicron Variant (B.1.1.529, BA.1) Spike RBD
Antigen
(SEQ ID NO: 14)
Amino Acid Sequence. Red Secretion Tag in bold font.
MPLLLLLPLLWAGALARVQPTESIVRFPNITNLCPFDEVFNATRFAS
VYAWNRKRISNCVADYSVLYNLAPFFTFKCYGVSPTKLNDLCFTNV
YADSFVIRGDEVRQIAPGQTGNIADYNYKLPDDFTGCVIAWNSNKLD
SKVSGNYNYLYRLFRKSNLKPFERDISTEIYQAGNKPCNGVAGFNCY
FPLRSYSFRPTYGVGHQPYRVVVLSFELLHAPATVCGPKKS
TNLVKNK.

In some embodiments, multimer antigens include a polypeptide or a nucleic acid encoding a polypeptide (mRNA) including two or more of RDB peptides represented by SEQ ID NOs: 2, 4, 6, 8, 10, 12, and 14, or a variant thereof having more than 90%, 91%, 92%, 93%, 94%, 95%, 96%, 97%, 98%, or 99% sequence identity to the amino acid sequence of SEQ ID NOs. 2, 4, 6, 8, 10, 12, and 14. In other embodiments, multimer antigens include a nucleic acid encoding two or more RDB peptides represented by SEQ ID NOs: 1, 3, 5, 7, 9, 11, and 13, or a variant thereof having more than 90%, 91%, 92%, 93%, 94%, 95%, 96%, 97%, 98%, or 99% sequence identity to the amino acid sequence of SEQ ID NOs. 1, 3, 5, 7, 9, 11, and 13.

An exemplary embodiment is a polypeptide or a nucleic acid encoding a polypeptide including a tPA signal peptide leading into the RBD of SARS-CoV-2 variant B.1.617.2 (“delta” variant; AA319-606) directly fused with the RBD of MERS-CoV (AA367-606) and followed by a Flag-tag, as depicted below (FIG. 1). An additional embodiment is a polypeptide encoding a tPA signal peptide leading into the RBD of SARS-CoV-2 variant B.1.1.7 (“alpha” variant; AA319-537) directly fused with the RBD of MERS-CoV (AA367-606) and followed by a Flag-tag.

Exemplary components of an immunogenic composition using the disclosed multimer approach can be, but are not limited to, RBDs of the Spike protein from the following strains:
SARS-CoV-2
   Wuhan.
   Variants of Concern (B.1.1.7, B.1.351, B.1.617.2, P1)
   Variants of Interest (B.1.525, B.1.526, B.1.617.1, C.37, B.1.621).
SARS-CoV-1
MERS-CoV
Seasonal HCoV (OC43, HKU1, NL63, 229E)

In preferred embodiments, the multimer antigens also include affinity tags enable purification and rapid detection of viable clones. Exemplary affinity tags include FLAG-tag having the amino acid sequence DYKDDDDK (SEQ ID NO: 15), His-tag having 6 or more histidine residues, Spot-tag having the amino acid sequence PDRVRAVSHWSS (SEQ ID NO: 16), C-tag having the amino acid sequence EPEA, and Strep-Tag having the amino acid sequence WSHPQFEK (SEQ ID NO: 17).

### B. Nanoparticles and Microparticles

In some embodiments, compositions of one or more heterogenous multimer antigens for eliciting immune responses against multiple coronaviruses include one or more particles for delivery into the body.

Appropriate delivery vehicles for the compounds are known in the art and can be selected to suit the particular antigen compositions. For example, in some embodiments, the composition is incorporated into or encapsulated by, or bound to, a nanoparticle, microparticle, microsphere, micelle, synthetic lipoprotein particle, or carbon nanotube.

For example, the compositions can be incorporated into a vehicle such as polymeric microparticles or polymeric nanoparticles which provide controlled release of the active agent(s). In some embodiments, release of the drug(s) is controlled by diffusion of the active agent(s) out of the particles and/or degradation of the polymeric particles by hydrolysis and/or enzymatic degradation.

In preferred embodiments, the compositions are incorporated into polymeric microparticles or polymeric nanoparticles that provide controlled release of the active agent(s), reduce rapid clearance from the system, and/or reduce rapid liver metabolism of the active agent(s).

Generally, the particles are formed from one or more polymers, lipids, or other suitable materials which encapsulate, are complexed with, or are otherwise associated with the one or more heterogenous multimer antigens. The particles shield the heterogenous multimer antigen from degradation or destruction prior to reaching the target site/cells within the body and thereby enhance the serum half-life and residence time of the heterogenous multimer antigen. In some embodiments, the particles include one or more targeting agents, for example, to deliver the heterogenous multimer antigen to any targeted site, e.g., specific cell types, specific organelles.

The particles permit and/or enhance the biological activity of the encapsulated or associated active agents. Typically, the particles protect the active agents to effectively prolong the residence time of the active agents in vivo. Therefore, the particles effectively increase the serum half-life of the active agent(s) in vivo as compared to the half-life of the heterogenous multimer antigens in the absence of a particle. In some embodiments, two different heterogenous multimer antigens or one heterogenous multimer antigen with another monomeric antigen are incorporated into the same particles.

### 1. Polymeric Nanoparticles

In some embodiments, the heterogenous multimer antigens are encapsulated within or complexed with a polymeric nanoparticle.

The particle can be a polymeric particle, a lipid particle, a solid lipid particle, an inorganic particle, or combinations thereof. For example, the particle can be a lipid-stabilized polymeric particle. In preferred embodiments the particle is a polymeric particle, a solid lipid particle, or a lipid-stabilized polymeric particle. The particle can include a polymeric particle formed from biodegradable polymers, non-biodegradable polymers, or a combination thereof. The polymeric particle core can be a biodegradable polymeric core in whole or in part.

### FDA Approved Biodegradable Polymers

In some embodiments, the heterogenous multimer antigens are encapsulated within and/or complexed with a polyhydroxy acid ester such as poly(lactic-co-glycolic acid) (PLGA), poly(lactic acid) (PLA), or poly(glycolic acid) (PGA), to form a particle with nanometer dimensions. Poly(lactic-co-glycolic acid) (PLGA), or (PLG), is a copolymer which is used in many Food and Drug Administration (FDA) approved therapeutic devices, owing to its biodegradability and biocompatibility. During polymerization, successive monomeric units (of glycolic or lactic acid) are linked together in PLGA by ester linkages, thus yielding a linear, aliphatic polyester as a product.

Other FDA approved polymers include polyanhydrides, polyorthoesters, polyhydroxyalkanoates, and some non-biodegradable polymers such as polymethacrylate and cyanomethacrylate. Particle includes polyhydroxyalkanoates are particularly suited for delivering to areas along the gastrointestinal tract and/or for systemic delivery via absorption through gastrointestinal tract follow enteral administration. In preferred embodiments, the particle includes polyhydroxyalkanoates.

In some embodiments, the particle includes the biodegradable polymer blended with, or covalently bound to one or more additional polymers. The additional polymers can be present within the inner core and/or outer surface of the particle upon formation, for example, blended with PLGA, or attached exclusively to the outside of the particle.

Polyalkylene oxide (PEO) polymers (also referred to as polyalkylenes, polyalkylene glycols, or polyalkylene oxides), are frequently bound to the surface of the polymers, or covalently bound to a hydrophobic biodegradable polymer, which self-assemble to form particles having the PEO polymers on the surface and the hydrophobic polymer in the core. A preferred PEO is polyethylene glycol (PEG).

### Other Biodegradable Polymers

In some embodiments the heterogenous multimer antigens are encapsulated within and/or complexed with biodegradable polymers. Exemplary biodegradable polymers include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Representative biodegradable polymers include polyamides, polycarbonates, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, modified celluloses, and acrylate polymers.

Excipients may also be added to the core polymer to alter its porosity, permeability, and or degradation profile.

### Hydrophilic Polymers

In some embodiments the heterogenous multimer antigens are encapsulated within and/or complexed with one or more hydrophilic polymers. Representative hydrophilic polymers include cellulosic polymers such as starch and polysaccharides: hydrophilic polypeptides and poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides): poly(hydroxy acids): poly(vinyl alcohol), and copolymers thereof.

### Hydrophobic Polymers

Representative hydrophobic polymers include polyhydroxyacids such as poly(lactic acid), poly(glycolic acid), and poly(lactic acid-co-glycolic acids): polyhydroxyalkanoates such as poly3-hydroxybutyrate or poly4-hydroxybutyrate: polycaprolactones: poly(orthoesters): polyanhydrides; poly(phosphazenes): poly(lactide-co-caprolactones): polycarbonates such as tyrosine polycarbonates: polyamides (including synthetic and natural polyamides), polypeptides, and poly(amino acids): polyesteramides: polyesters: poly(dioxanones): poly(alkylene alkylates); hydrophobic polyethers: polyurethanes: polyetheresters: polyacetals: polycyanoacrylates: polyacrylates: polymethylmethacrylates: polysiloxanes: poly(oxyethylene)/poly (oxypropylene) copolymers: polyketals: polyphosphates: polyhydroxyvalerates: polyalkylene oxalates: polyalkylene succinates: poly(maleic acids), as well as copolymers thereof.

### Amphiphilic Polymers

Representative amphiphilic polymers include block copolymers of any of the hydrophobic and hydrophilic polymers described above. Amphiphilic compounds also include phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer).

### PEGylation

As noted above, modification of the surface of the particles to increase available hydroxyl groups, typically by incorporation into or onto the surface PEO polymers, can be used to decrease clearance rates from the blood and to enhance cellular or tissue uptake. Methods for surface modification of the polymeric particles, or incorporation of PEO polymers are known to those skilled in the art.

### 2. Lipidic Particles

In some embodiments, the particle is a lipid particle, liposome, or micelle, or includes a lipid core. Lipid particles and lipid nanoparticles are known in the art. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

Representative neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI): glycolipids: sphingophospholipids such as sphingomyelin and sphingoglycolipids (also known as 1-ceramidyl glucosides) such as ceramide galactopyranoside, gangliosides and cerebrosides: fatty acids, sterols, containing a carboxylic acid group for example, cholesterol.

Representative cationic lipids include, but are not limited to, N-[1-(2,3-dioleoyloxy) propyl]-N,N,N-trimethyl ammonium salts, referred to as TAP lipids, for example, methylsulfate salt. Representative TAP lipids include, but are not limited to, DOTAP (dioleoyl-), DMTAP (dimyristoyl-), DPTAP (dipalmitoyl-), and DSTAP (distearoyl-). Representative cationic lipids in the liposomes include, but are not limited to, dimethyldioctadecyl ammonium bromide (DDAB), 1,2-diacyloxy-3-trimethylammonium propanes, N-[1-(2,3-dioloyloxy) propyl]-N,N-dimethyl amine (DODAP), 1,2-diacyloxy-3-dimethylammonium propanes, N-[1-(2,3-dioleyloxy) propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol): 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl) diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9 (Z)-octadecenoyloxy)ethyl]-2-(8 (Z)-heptadecenyl-3-(2-hydroxyethyl) imidazolinium chloride (DOTIM), and 1-[2-(hexadecanoyloxy)ethyl]-2-pentadecyl-3-(2-hydroxyethyl) imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

### Micelles

In some embodiments, the particle or particle core is a lipid micelle. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

### Liposomes

In some embodiments, the particle or particle core is a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate targeted agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Representative alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

In one embodiment, liposomes are prepared from long chain fatty acids and phytosterol formulations.

### Solid Lipid Particles

In some embodiments, the particle is a solid lipid particle, or includes a solid lipid core. Solid lipid particles present an alternative to the colloidal micelles and liposomes. Solid lipid particles are typically submicron in size, i.e., from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Representative solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Representative solid lipids can include cetyl palmitate or beeswax. Cyclodextrin can also be used.

### 3. Inorganic Particles

In some embodiments, the particle is formed from, or includes a core formed from an inorganic particle such as metal, metal oxide or semiconductor particles. The particle can be a metal nanoparticle, a semiconductor nanoparticle, or a core-shell nanoparticle. Inorganic particles and inorganic nanoparticles can be formulated into a variety of shapes such as rods, shells, spheres, and cones. The inorganic particle may have any dimension. The inorganic particle can have a greatest dimension less than 1 micron, from about 10 nm to about 1 micron, from about 10 nm to about 500 nm, or from 10 nm to about 250 nm.

The inorganic particle or particle core can contain a metal oxide. Metal oxides of any of the above metals are contemplated. Suitable metal oxides can include metal oxides that contain one or more of the following metals: titanium, scandium, iron, tantalum, cobalt, chromium, manganese, platinum, iridium, niobium, vanadium, zirconium, tungsten, rhodium, ruthenium, copper, zinc, yttrium, molybdenum, technetium, palladium, cadmium, hafnium, rhenium and combinations thereof. Suitable metal oxides can include cerium oxides, platinum oxides, yttrium oxides, tantalum oxides, titanium oxides, zinc oxides, iron oxides, magnesium oxides, aluminum oxides, iridium oxides, niobium oxides, zirconium oxides, tungsten oxides, rhodium oxides, ruthenium oxides, alumina, zirconia, silicone oxides such as silica-based glasses and silicon dioxide, or combinations thereof. The metal oxide can be non-biodegradable. The metal oxide can be a biodegradable metal oxide. Biodegradable metal oxides can include silicon oxide, aluminum oxide and zinc oxide.

### Hybrid Particles

In some embodiments, the particle or particle core is a hybrid particle. Hybrid particle, as used herein, refers to a particle that combines the features of two or more of polymeric particles, lipid particles, and inorganic particles. Examples of hybrid particles can include polymer-stabilized liposomes, polymer-coated inorganic particles, or lipid-coated polymeric particles. The hybrid particle can contain a polymeric inner region, a lipid inner region, or an inorganic inner region. The hybrid particle can contain a polymer outer layer, a lipid outer layer, or an inorganic outer layer.

### 4. Dendrimeric Particles

In some embodiments, the particle or particle core is a dendrimer. Dendrimers are three-dimensional, hyperbranched, monodispersed, globular and polyvalent macromolecules comprising a high density of surface end groups. The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Suitable dendrimers scaffolds that can be used include poly(amidoamine), also known as PAMAM, or STARBURST™ dendrimers: polypropylamine (POPAM), polyethyleneimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In general, agent is conjugated in a mass ratio of agent to dendrimer of between 0.1:1 and 4:1, inclusive.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and agents.

### C. Adjuvants

The disclosed compositions include one or more adjuvants. Adjuvants are known.

Exemplary adjuvants include, but are not limited to, aluminum hydroxide, aluminum phosphate, emulsion adjuvants, MF59, and AS03. LR agonists have been extensively studied as vaccine adjuvants. CpG, Poly I:C, glucopyranosyl lipid A (GLA), and resiquimod (R848) are agonists for TLR9, TLR3, TLR4, and TLR7/8, respectively.

Oil-Emulsion Adjuvants include squalene-water emulsions, such as MF59 (5% Squalene, 0.5% Tween 80, and 0.5% Span 85, formulated into submicron particles using a microfluidizer). See, e.g., WO90/14837, and, Podda, Vaccine 19:2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s-n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below). Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also comprise a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739. Optionally, the ISCOMS can be devoid of additional detergent. See WO00/07621. A description of the development of saponin based adjuvants can be found at Barr, et al., “ISCOMs and other saponin based adjuvants”, Advanced Drug Delivery Reviews 32:247-27, 1998. See also Sjolander, et al., “Uptake and adjuvant activity of orally delivered saponin and ISCOM vaccines”, Advanced Drug Delivery Reviews 32:321-338, 1998.

Bioadhesives and mucoadhesives can also be used as adjuvants. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some embodiments, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19:109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27:571-577, 2002 and Jones, “Resiquimod 3M”, Curr Opin Investig Drugs 4:214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

### C. Pharmaceutically Acceptable Carriers

In some embodiments, the compositions include one or more pharmaceutically acceptable carriers, or excipients, or preservatives. Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable carriers include, but are not limited to, buffers, diluents, preservatives, binders, stabilizers, a mixture or polymer of sugars (lactose, sucrose, dextrose, etc.), salts, and combinations thereof.

The compositions may be administered in combination with one or more physiologically or pharmaceutically acceptable carriers, thickening agents, co-solvents, adhesives, antioxidants, buffers, viscosity, and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

In a preferred embodiment, the compositions are administered in an aqueous solution, by parenteral injection or infusion. The formulation may also be in the form of a suspension or emulsion. In general, pharmaceutical compositions are provided including effective amounts of the composition, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as antioxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

In some embodiments, the pharmaceutical composition for cells is a saline solution, preferably a buffered saline solution phosphate buffered saline or sterile saline, or tissue culture medium.

### III. Methods of Using

Methods for preventing or treating one or more symptoms of coronavirus infection in the subject are described. The methods administer an effective of the disclosed formulations to a subject in need thereof, to elicit an immune response such as a neutralizing immune response against a combination of viruses such as a combination of members of the beta coronavirus (CoV) family, such as SARS-CoV-2, MERS-CoV, or other Human (H)CoVs. Coronaviruses are named for the crown-like spikes on their surface. There are four main sub-groupings of coronaviruses, known as alpha, beta, gamma, and delta. The common human coronaviruses include 229E (alpha coronavirus), NL63 (alpha coronavirus), OC43 (beta coronavirus), HKU1 (beta coronavirus). Other human coronaviruses include MERS-CoV (the beta coronavirus that causes Middle East Respiratory Syndrome, or MERS), SARS-CoV (the beta coronavirus that causes severe acute respiratory syndrome, or SARS), and SARS-CoV-2 (the novel coronavirus that causes coronavirus disease 2019, or COVID-19).

### A. Subjects to be Treated

A subject in need of treatment is a subject having or at risk of having an infection e.g., a subject having or at risk of contracting a viral infection. The methods are particularly suited for those at risk of exposure to one or more respiratory pathogens such as SARS-CoV-2. Thus, in some embodiments, the subject has not experienced any symptoms from COVID but is at risk of doing so.

A positive SARS-CoV-2 viral test (i.e., reverse transcription polymerase chain reaction [RT-PCR] test or antigen test) or serologic (antibody) test can help assess for current or previous infection.

In some embodiments, the methods provide an effective amount of the composition to prevent one or more symptoms of coronavirus infection in the subject, for example, reducing or preventing one or more symptoms or physiological markers of severe acquired respiratory syndrome (SARS) in a subject. Exemplary symptoms of COVID-19 include cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Therefore, in some embodiments, the methods prevent or reduce one or more of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

In some embodiments, the methods reduce or prevent infection by the causative viral disease COVID-19 in a subject. In other embodiments, the methods prevent or reduces the invading viral pathogens in getting inside and/or proliferating in one or more targeting cells.

In some embodiments, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

The subject can be about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger. In some embodiments, the subject is a young adult between the ages of about 20 years and about 50 years (e.g., about 20, 25, 30, 35, 40, 45 or 50 years old). In some embodiments, the subject is an elderly subject about 60 years old, about 70 years old, or older (e.g., about 60, 65, 70, 75, 80, 85 or 90 years old).

### B. Methods of Administration

The compositions are generally administered to a subject in an effective amount. As used herein the term “effective amount” means a dosage sufficient to inhibit, or prevent one or more infections, or symptoms of a disease or to otherwise provide a desired pharmacologic and/or physiologic effect. The precise dosage will vary according to a variety of factors such as subject-dependent variables (e.g., age, immune system health, etc.), the specific variant of virus, and the treatment being affected.

The pharmaceutical compositions can be for administration by parenteral (intramuscular, intraperitoneal, intravenous, or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In preferred embodiments, the compositions are administered locally, for example by intranasal administration. Typically, local administration causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

In one embodiment, the method includes administration via a nebulizer to a subject of an effective amount of the disclosed composition.

In the most preferred embodiments, the immunizing virus is delivered peripherally by intranasally or by intramuscular injection, and the booster formulation is delivered by local injection.

### C. Treatment Regimens

A treatment regimen can include one or multiple administrations of the compositions and formulations thereof for achieving a desired physiological change, including administering to an animal, such as a mammal, especially a human being, an effective amount of the compositions to treat the disease or symptom thereof, or to produce the physiological change. In preferred embodiments, the desired physiological change is the reduction in the amount of syncytial formation and lung damage in the subject.

### 1. Dosage and Effective Amounts

A therapeutically effective amount of the immunogenic formulations used in the treatment of diseases and disorders associated with coronavirus infection are typically sufficient to reduce or alleviate one or more symptoms of the diseases and disorders associated with coronavirus infection. Symptoms of diseases and disorders associated with coronavirus infection may be cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Accordingly, the amount of immunogenic formulations can be effective to, for example, treat or prevent one or more symptoms of a coronavirus infection. Preferably the immunogenic formulations are delivered topically to the mucosal surface of the lung. Preferably the immunogenic formulations do not target or otherwise modulate other metabolic processes or metabolic products. In some embodiments, the immunogenic formulations are administered in an effective amount to reduce or alleaviate coronavirus infection, or one or more diseases or disorders associated with coronavirus infection in a subject at risk of exposure to SAR-Cov-2 virus.

The immunogenic formulations are administered to a subject in need thereof such as a human subject. The subject preferably had been primed, either by an infection with the pathogen or an immunization against the pathogen, either of which result in a balanced T cell and B cell immune response to the pathogen. In some embodiments, the dosage units include an effective amount for inducing or stimulating a protective T cell and/or B cell immune response to the pathogen.

The precise dosage will vary according to a variety of factors such as subject-dependent variables (e.g., age, immune system health, clinical symptoms etc.). Persons of ordinary skill can determine optimum dosages, dosing methodologies and repetition rates, which may vary depending on the relative potency of individual vaccines, and can generally be estimated based on EC50s found to be effective in ex vivo assay and in in vivo animal models.

The disclosed immunogenic formulations produce prophylactically—and/or therapeutically efficacious levels, concentrations and/or titers of antigen-specific antibodies in the blood or serum of a subject to whom it is administered. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc. In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

### 2. Controls

The therapeutic result of the compositions including a single dose or a plurality of doses of a composition including (i) an RNA molecule encoding antigens from multiple coronaviruses or (ii) a fusion peptide containing antigens from multiple coronaviruses (heterogeneous antigens) can be compared to a control. Suitable controls are known in the art and include, for example, untreated cells or an untreated subject. A typical control is a comparison of a condition or symptom of a subject prior to and after administration of the composition. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### D. Methods for Determining Immune Responses

Methods for determining immune responses are known in the art. In some embodiments, viral lesions can be examined to determine the occurrence of an immune response to the virus and/or the antigen. In some embodiments, in vitro assays may be used to determine the occurrence of an immune response. Examples of such in vitro assays include ELISA assays and cytotoxic T cell (CTL) assays. In some embodiments, the immune response is measured by detecting and/or quantifying the relative amount of an antibody, which specifically recognizes an antigen in the sera of a subject who has been treated by administering the heterogeneous multimer antigen, relative to the amount of the antibody in an untreated subject.

Techniques for the assaying antibodies and antibody filters in a sample are known in the art and include, for example, sandwich assays, ELISA and ELISpot. Polyclonal sera are relatively easily prepared by injection of a suitable laboratory animal with an effective amount of the immune effector, or antigenic part thereof, collecting serum from the animal and isolating specific sera by any of the known immunoadsorbent techniques. Antibodies produced by this method are utilizable in virtually any type of immunoassay.

The use of monoclonal antibodies in an immunoassay is preferred because of the ability to produce them in large quantities and the homogeneity of the product. The preparation of hybridoma cell lines for monoclonal antibody production derived by fusing an immortal cell line and lymphocytes sensitized against the immunogenic preparation can be achieved by techniques which are well known to those who are skilled in the art. In other embodiments, ELISA assays may be used to determine the level of isotype specific antibodies using methods known in the art.

Mature B cells can be measured in immunoassays, for example, by cell surface antigens including CD19 and CD20 with monoclonal antibodies labeled with fluorochromes or enzymes may be used to these antigens. B cells that have differentiated into plasma cells can be enumerated by staining for intracellular immunoglobulins by direct immunofluorescence in fixed smears of cultured cells.

Immunoglobulin Production Assay: B cell activation results in small, but detectable, quantities of polyclonal immunoglobulins. Following several days of culture, these immunoglobulins may be measured by radioimmunoassay or by enzyme-linked immunosorbent assay (ELISA) methods.

B cells that produce immunoglobulins can also be quantified by the reversed hemolytic plaque assay. In this assay, erythrocytes are coated with goat or rabbit anti-human immunoglobulins. These immunoglobulins are mixed with the activated immunoglobulin-producing lymphocytes and semisolid agar, and complement is added. The presence of hemolytic plaques indicates that there are immunoglobulin-producing cells.

Addback Assays: When added to fresh peripheral blood mononuclear cells, autologous ex vivo activated cells exhibit an enhanced response to a “recall” antigen, which is an antigen to which the peripheral blood mononuclear cells had previously been exposed. Primed or stimulated immune cells should enhance other immune cells response to a “recall” antigen when cultured together. These assays are termed “helper” or “addback” assays. In this assay, primed or stimulated immune cells are added to untreated, usually autologous immune cells to determine the response of the untreated cells. The added primed cells may be irradiated to prevent their proliferation, simplifying the measurement of the activity of the untreated cells. These assays may be particularly useful in evaluating cells for blood exposed to virus. The addback assays can measure proliferation, cytokine production, and target cell lysis as described herein.

The above-described methods and other additional methods to determine an immune response are known in the art.

### IV. Methods of Making

Methods of making a single RNA molecule or recombinant protein include (i) an RNA molecule encoding antigens from multiple coronaviruses or (ii) a fusion peptide containing antigens from multiple coronaviruses (heterogeneous antigens) are described.

The disclosed methods minimize the number of RNA constructs included in a vaccine formulation, allow for lower dosage, particularly in a replicon vaccine, as the nonstructural components of the replicon typically contribute a greater portion of the total mass of the RNA than the encoded antigens.

Methods of making a single RNA molecule encoding antigens from multiple coronaviruses are described. In some embodiments, the RNA construct includes a signal peptide and two RBD segments from different betacoronaviruses, optionally a purification/affinity tag such as FLAG tag. In one embodiment, the RNA construct is prepared as shown in FIG. 1.

Fusion proteins including the heterogeneous antigens as disclosed herein, can be made using methods knowing the art for engineering expression of fusion proteins/peptides.

Fusion proteins can be made for example, by chemical synthesis, and more preferably, by recombinant production in a host cell. To recombinantly produce a fusion protein including the heterogeneous antigens as disclosed herein, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a fusion protein including the heterogeneous antigens as disclosed herein. Regulatory sequences (also referred to herein as expression control sequences) typically do not encode a gene product, but instead affect the expression of the nucleic acid sequences to which they are operably linked. The nucleotide sequences encoding the fusion protein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector is preferably an expression vector in which the DNA sequence encoding the fusion protein is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g., transcription initiates in a promoter and proceeds through the DNA sequence coding for the fusion protein. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Expression vectors for use in expressing the fusion protein will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell. Examples of suitable promoters for directing the transcription of the DNA in mammalian cells are the SV40 promoter (Subramani et al., Mol. Cell. Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

The expressed tagged or fusion proteins produced by the cells may be recovered from the culture medium by conventional procedures including separating the host cells from the medium by centrifugation or filtration, releasing the fusion protein by mechanical cell disruption, such as ultrasonication or pressure, precipitating the proteinaceous components of the supernatant or filtrate by means of a salt, e.g., ammonium sulphate. After sonication a suitable concentration of NaCl can be added to further decrease the ability of host cell contaminants to bind to the cation exchange matrix. After cation-exchange chromatography the fusion protein may be eluted in a salt gradient and eluate fractions containing the fusion protein are collected. In some preferred embodiments, fusion protein is captured from lysate through its His tag.

Particularly preferred embodiments are exemplified below. The constructs used in the disclosed compositions can be made as exemplified below for the SARS-CoV-2 delta-MERS-CoV RBD dimer construct.

### V. Kits

Kits are also disclosed. The kit can include a single dose or a plurality of doses of a composition including (i) an RNA molecule encoding antigens from multiple coronaviruses or (ii) a fusion peptide containing antigens from multiple coronaviruses (heterogeneous antigens), or pharmaceutical formulation thereof, and instructions for administering the compositions. Specifically, the instructions direct that an effective amount of the composition be administered to an individual at risk of exposure to one or more respiratory pathogens such as severe acute respiratory syndrome (SARS) virus. The composition can be formulated as described above with reference to a particular treatment method and can be packaged in any convenient manner.

The present invention will be further understood by reference to the following non-limiting examples.

### EXAMPLES

### Example 1: SARS-CoV-2 Delta-MERS-CoV RBD Dimer Construct and In Vitro Validation

### Materials and Methods

BHKs in 12-well dishes were transfected with 1.5 μg of replicon RNA encoding a fusion dimer of SARS-CoV-2 variant B.1.617.2 and MERS-CoV RBD subunits using a commercial transfection reagent (TransIT-mRNA, Mirus Bio). The next day, cells were harvested, and lysates were separated by SDS-PAGE, transferred to PVDF membranes, and analyzed by immunoblotting. Membranes were probed with a mouse monoclonal antibody against SARS-CoV-2 RBD (R&D Systems; left) or a rabbit polyclonal antibody against MERS-CoV Spike protein (eEnzyme, LLC; right) diluted at 1:1000. Appropriate secondary antibody (mouse and rabbit, respectively) conjugated to HRP was used to detect the immunocomplexes. Membranes were stripped and probed for GAPDH using a monoclonal antibody as loading control (ThermoFisher Scientific). The predicted mass of the fusion protein is ˜54 kDa.

### Results

As proof-of-concept, a DNA fragment encoding a SARS-CoV-2 delta-MERS-CoV RBD dimer construct was produced with overhangs allowing for direct cloning into an RNA replicon expression cassette by excision of the PflfI/MfeI restriction fragment downstream of the Venezuelan equine encephalitis virus (VEEV) replicon nonstructural genes by In-Fusion Cloning (Takara Bio). The derived expression vector was used as the DNA template for synthesis of Replicon RNA by in vitro T7-based transcription and vaccinia capping (Hongene).

FIG. 1 shows the schematic design of an exemplary synthetic polypeptide, N-terminus to the left and C-terminus to the right, including a signal peptide and two RBD segments from different betacoronaviruses. A C-terminal FLAG tag is included. This synthetic protein can be manufactured as recombinant protein by an expression system such as CHO or HEK293 cells. A nucleic acid molecule such as DNA or RNA can be manufactured to encode this synthetic protein.

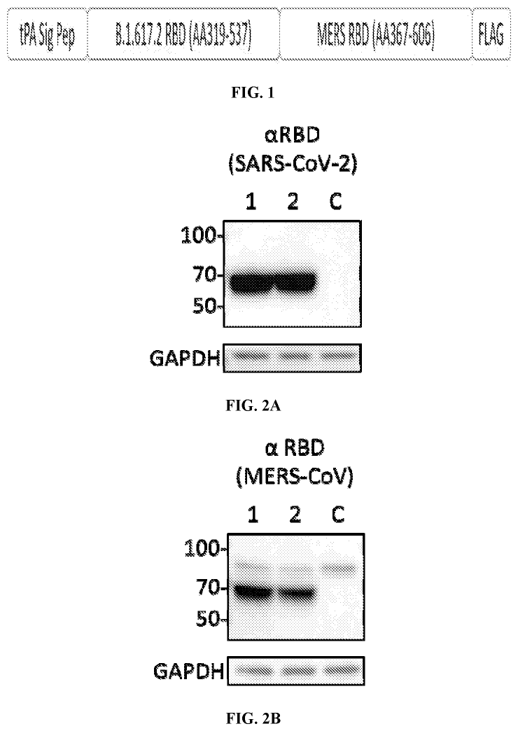

Following purification by lithium chloride precipitation, RNA was tested for expression of SARS-CoV-2 delta variant RBD and the MERS-CoV RBD in vitro using BHK cells. Immunoblot experiment was performed on the lysates of cells transfected with RNA encoding the polypeptide depicted in FIG. 1 (B.1.617.2-MERS RBD DIMER). The same lysates were immobilized on a PVDF membrane and probed with antibody specific for SARS-CoV-2 (FIG. 2A) or MERS-CoV (FIG. 2B). The presence of bands at the identical molecular weight in each blot indicates that the synthetic polypeptide expressed upon translation of the RNA carries dual antigenicity that is recognized by sera specific to two different betacoronaviruses simultaneously.

### Example 2: In Vivo Administration of SARS-CoV-2 Delta-MERS-CoV RBD Dimer Construct Induces Rapid Humoral Responses

### Materials and Methods

Day 10 serum samples from Trivalent RNA vaccinated mice (2 μg dose) were analyzed against the three vaccine target subunits: SARS-CoV-2 Beta RBD, SARS-CoV-2 Delta RBD, and MERS-CoV RBD. The Trivalent RNA vaccine was composed of one RNA molecule encoding the Delta/MERS dimer, and another encoding the full-length SARS-CoV-2 variant B.1.351 (Beta) full-length Spike protein. All assays were executed separately. Briefly, plates were coated with 1 μg/mL of respective protein in PBS overnight at 4 C. Serum samples were plated with serial dilutions ranging from 1:25 to 1:51200 and incubated for 1 hour at room temperature. Goat anti-Mouse IgG HRP detection antibody was used, and plates were developed with TMB followed by Stop Solution (H₂SO₄).

### Results

Upon validation, RNA was formulated with a dendrimeric delivery molecule and injected into C57BL/6 mice at a 5-μg dose. Two weeks post-vaccination, serum samples were collected and analyzed via direct ELISA against SARS-CoV-2 delta RBD and MERS-CoV RBD.

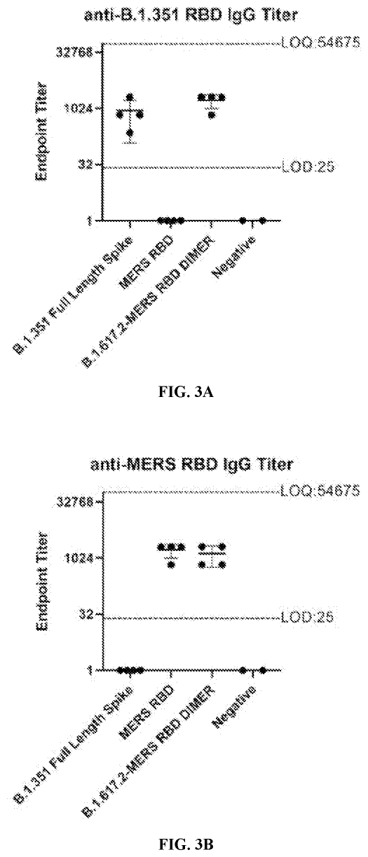

Serum samples from mice vaccinated with the SARS-CoV-2 delta-MERS-CoV RBD dimer exhibited positive antibody titers against both the SARS-CoV-2 delta BRD and the MERS-CoV RBD, and at comparable levels to mice vaccinated in parallel with comparable RNAs encoding full length SARS-CoV-2 delta Spike protein in the anti-SPIKE ELISA or with MERS-CoV RBD only in the anti-MERS ELISA (FIGS. 3A-3B).

FIG. 3A is the result of immunization of mice with an RNA vaccine encoding the polypeptide depicted in FIG. 1 (B.1.617.2-MERS RBD DIMER) or alternative constructs as controls. In this experiment, serum from the immunized mice, (4 animals per group, 2 animals in the Negative control group) were tested for IgG antibody titer against SARS-CoV-2 RBD (from variant strain B.1.351). Animals immunized with RNA encoding SARS-CoV-2 Spike protein only (control vaccine, B.1.351 Full Length Spike) exhibited titers above the limit of detection (LOD). Conversely, mice immunized with another control RNA encoding MERS RBD alone did not exhibit SARS-CoV-2-specific titers. The B.1.617.2-MERS RBD DIMER successfully induced SARS-CoV-2-specific titers. In FIG. 3B, serum from the same experiment was tested for IgG antibody titer against MERS-CoV RBD. In this case, the control SARS-CoV-2 vaccine B.1.351 Full Length Spike failed to elicit antibody titers. The MERS RBD control vaccine worked. The B.1.617.2-MERS RBD DIMER successfully induced MERS RBD-specific titers. Together, this data show that the B. 1.617.2-MERS RBD DIMER construct depicted in FIG. 1 was the only construct capable of inducing simultaneous antibody responses against the two divergent species of betacoronavirus.

Shown above are results of a ‘dimer’ composed of the mRNA SARS-CoV-2 delta RBD and MERS-CoV RBD, any combination of coronavirus RBDs can replace either component of the ‘dimer’ with this approach. For example, the order of the RBDs could be switched in the example, with the MERS-CoV RBD as the N-terminal half of the dimer and the SARS-CoV-2 RBD as the C-terminal half. In addition, combinations of different SARS-CoV-2 variants could be used within a construct, as well as SARS-CoV-1 or seasonal HCoVs following the ‘multimer’ approach. As such, adding on a third RBD to the construct, resulting in a ‘trimeric’ structure.

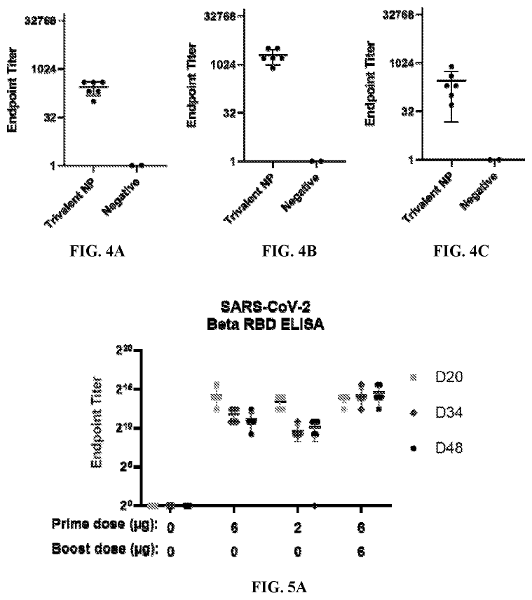

FIGS. 4A-4C are results of immunization of mice with a mixture of the B.1.617.2-MERS RBD dimer (FIG. 1) RNA and an additional vaccine RNA encoding the SARS-CoV-2 Spike protein only, analyzed by ELISA. These two RNA molecules were co-formulated for administration as a single nanoparticle product (“Trivalent NP”). The serum from immunized mice reacted with at least two variants of SARS-CoV-2 (Beta and Delta) as well the unrelated MERS-CoV, demonstrating a breadth of response greater than any single betacoronavirus strain.

### Example 3: In Vivo Prime/Boost Regimen of SARS-CoV-2 Delta-MERS-CoV RBD Dimer Construct Induces Long-Term Humoral Responses

### Materials and Methods

Day 20, 34, and 48 serum samples from Trivalent RNA vaccinated mice (n=6) at 2 or 6 μg doses were analyzed against the three vaccine target subunits: SARS-CoV-2 Beta RBD, SARS-CoV-2 Delta RBD, and MERS-CoV RBD. All assays were executed separately. The Trivalent RNA vaccine was composed of one RNA molecule encoding the Delta/MERS dimer, and another encoding the full-length SARS-CoV-2 variant B.1.351 (Beta) full-length Spike protein.

### Results

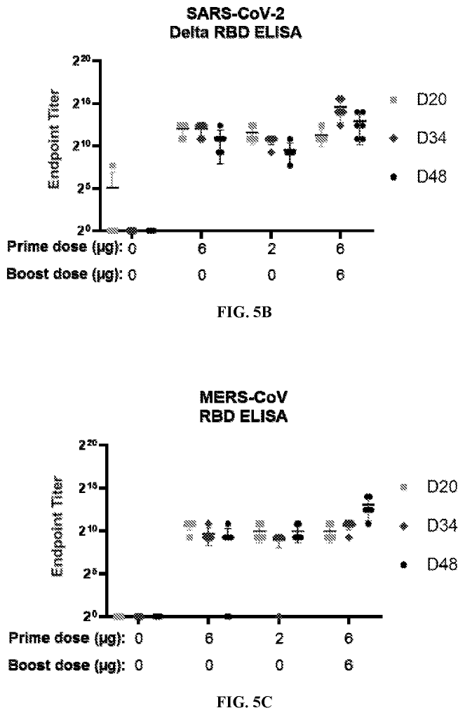

FIGS. 5A-5C show that the highest sustained titers against two SARS-CoV-2 variants and MERS-CoV induced by the Trivalent NP RNA vaccine (see the Day 48 titers) is achieved by priming and boosting mice with 6 μg of the vaccine that contains the exemplary B.1.617.2-MERS RBD dimer.

### Discussion

A recent study reported a SARS-CoV-2 homodimer having two identical receptor binding domains (RBDs) (Pan X et al., Cell Discov. 2021; 7:82). The homodimer was produced by encoding monomers that dimerize post-translationally, which are then proteolytically digested after manufacture. This is far more onerous manufacturing to produce a vaccine that has less breadth than the simple approach carried out in the current study. Another study showed betacoronavirus dimers with two identical RBDs expressed in tandem (Dai L et al., 2020, Cell 182, 722-733). However, no reactivity to other virus strains or species shown for a single product, thus only capable of protecting against one species.

Other studies have also attempted eliciting immune response against RBDs. Chen et al. (Chen R et al., Adv. Sci. 2022, 9, 2105378) reported a vaccine against multiple SARS-CoV-2 variants and included three different RBD proteins individually that were arrayed on a ferritin nanoparticle scaffold. This production is onerous, and it only showed protection against a single species of coronavirus. Cohen et al. (Cohen A et al., Science. 2022 Jul. 5: eabq0839) report a ‘mosaic RBD’ nanoparticle. This also required the production of multiple individual RBD recombinant proteins from different viruses in one expression system, followed by conjugation of these proteins onto a 2-keto-3-deoxy-phosphogluconate (KDPG) aldolase enzyme-derived nanoparticle scaffold which in turn had to be produced in a different expression system. Unlike the heterogeneous multimer antigen shown here, they did not show protection across distantly related coronaviruses such as SARS-CoV-2 and MERS-CoV.

The method of inducing multi-virus immune responses in use for about 80 years (e.g., multivalent flu, 1942 onward) has been to mix multiple antigenic polypeptides into single formulations. The current study provides a single polypeptide with multi-antigenicity suitable for large-scale manufacturing. The multi-virus vaccines described here provide a much simpler manufacturing procedure than producing one formulation for each target antigen or virus.

1. An immunogenic composition comprising a polypeptide of or a nucleic acid encoding a polypeptide and optionally an adjuvant,
wherein the polypeptide comprises a heterogeneous multimer antigen with antigens derived from two or more strains of the coronavirus family.

2. The immunogenic composition of claim 1, wherein: (a) the nucleic acid is an mRNA, a replicon RNA, or DNA encoding the heterogeneous multimer antigen; (b) the polypeptide is a recombinant protein comprising the heterogeneous multimer antigen; and/or (c) the heterogeneous multimer antigen further comprises a signal peptide; optionally, wherein the heterogeneous multimer antigen further comprises an affinity tag.

3. (canceled)

4. The immunogenic composition of claim 1, wherein the heterogeneous multimer antigen comprises a first receptor-binding domain (RBD) of the spike protein of a first coronavirus and a second RBD of the spike protein of a second coronavirus, optionally a linker between the first and the second RBDs.

5. (canceled)

6. The immunogenic composition of claim 2, wherein the signal peptide is tissue plasminogen activator (tPA) signal sequence, or an extended tPA, or a signal peptide derived from a full-length coronavirus spike protein.

7. The immunogenic composition of claim 2, wherein the signal peptide is tPA signal sequence.

8. (canceled)

9. The immunogenic composition of claim 8, wherein the affinity tag is selected from the group consisting of FLAG-tag having the amino acid sequence DYKDDDDK (SEQ ID NO: 15), His-tag having 6 or more histidine residues, Spot-tag having the amino acid sequence PDRVRAVSHWSS (SEQ ID NO:16), C-tag having the amino acid sequence EPEA, and Strep-Tag having the amino acid sequence WSHPQFEK (SEQ ID NO:17).

10. The immunogenic composition of claim 8, wherein the affinity tag is FLAG-tag having the amino acid sequence DYKDDDDK (SEQ ID NO:15).

11. The immunogenic composition of claim 5, wherein the heterogeneous multimer antigen comprises a signal peptide at the N-terminus and an affinity tag at the C-terminus.

12. The immunogenic composition of claim 5, wherein the heterogeneous multimer antigen comprises a signal peptide at the N-terminus, followed by the first RBD of the spike protein of the first coronavirus, optionally a linker, and the second RBD of the spike protein of the second coronavirus and an affinity tag at the C-terminus.

13. The immunogenic compositions of claim 1, wherein the first and second coronavirus are selected from the group consisting of SARS-CoV-2, SARS-CoV-1, MERS-CoV, HCoV-OC43, HCoV-HKU1, HCoV-NL63, and HCoV-229E.

14. The immunogenic compositions of claim 1, wherein the first and second coronavirus are different strains of coronaviruses, optionally, wherein the first coronavirus is SARS-CoV-2 strain and second coronavirus is MERS-CoV strain.

15. (canceled)

16. The immunogenic compositions of claim 1, wherein the first coronavirus is a variant of SARS-CoV-2 selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

17. The immunogenic compositions of claim 1, wherein the first and the second RBDs comprise the amino acid sequence selected from the group consisting of SEQ ID NOs: 2, 4, 6, 8, 10, 12, 14, and a variant thereof having more than 90%, 91%, 92%, 93%, 94%, 95%, 96%, 97%, 98%, or 99% sequence identity to the amino acid sequence of SEQ ID NOs. 2, 4, 6, 8, 10, 12, and 14.

18. The immunogenic compositions of claim 1, wherein the first RBD comprises the amino acid sequence of SEQ ID NO: 12, and the second RBDs comprises the amino acid sequence of SEQ ID NO: 4.

19. A pharmaceutical formulation comprising the immunogenic composition of claim 1, and one or more pharmaceutically acceptable carrier.

20. The pharmaceutical formulation of claim 19, wherein the immunogenic composition is encapsulated within and/or associated with a delivery vehicle that increases the serum half-life of the immunogenic composition as compared to the serum half-life of the same amount of the immunogenic composition alone.

21. The pharmaceutical formulation of claim 20, wherein the delivery vehicle is a nanoparticle or microparticle selected from the group consisting of a liposome, a polymeric particle, a virus-like-particle, a dendrimeric particle, and a protein nanostructure; optionally, the delivery vehicle is a dendrimeric particle.

22. (canceled)

23. The pharmaceutical formulation of claim 19 formulated for intranasal or by intramuscular administration.

24. A method of eliciting an immune response against multiple coronaviruses in a subject in need thereof, comprising administering to the subject an effective amount of the pharmaceutical formulation of claim 19.

25. The method of claim 24, wherein the pharmaceutical formulation is administered by intranasally or by intramuscular injection and/or wherein the pharmaceutical formulation is administered to the subject in an amount effective to elicit an antibody response against two or more strains of coronaviruses.

26. (canceled)
